(19) United States
(12) Patent Application Publication
(10) Pub. No.: US 2020/0181709 A1
(43) Pub. Date: Jun. 11, 2020

(54) METHODS OF DETECTION AND TREATMENT OF UROTHELIAL CANCER

(71) Applicant: THE JOHNS HOPKINS UNIVERSITY, Baltimore, MD (US)

(72) Inventors: Jong Chul Park, McLean, VA (US); Leonel Francisco Maldonado Gonzalez, Baltimore, MD (US); Mariana Brait Rodrigues De Oliveira, Baltimore, MD (US); Mohammad O. Hoque, Towson, MD (US)

(21) Appl. No.: 15/737,228

(22) PCT Filed: Jun. 14, 2016
(86) PCT No.: PCT/US2016/037425

§ 371 Date: Dec. 15, 2017

Related U.S. Application Data
(60) Provisional application No. 62/175,897, filed on Jun. 15, 2015.

(51) Int. Cl. C12Q 1/6886; A61K 31/706; A61K 31/7068; A61K 31/164; A61P 35/00

(52) U.S. Cl. CPC C12Q 1/6886; A61K 31/706; A61K 31/7068; C12Q 2600/106; A61P 35/00; C12Q 2600/154; A61K 31/164

(57) ABSTRACT

The invention provides methods for detecting a cellular proliferative disorder (e.g., urothelial cancer) in a subject by assessing the methylation status of the CCND2, CCNA1 or CALCA promoter in a nucleic acid sample. The methods of the invention are useful for diagnostic, prognostic as well therapeutic regimen predictions.

### CROSS-REFERENCE TO RELATED APPLICATIONS

This application claims benefit of priority under 35 U.S.C. § 119(e) of U.S. Ser. No. 62/175,897, filed Jun. 15, 2015, the entire contents of which is incorporated herein by reference in its entirety.

### FIELD OF THE INVENTION

The invention relates generally to methods for diagnosing and treating cancer and more specifically to methods for detecting, diagnosing, providing prognosis for and treating urothelial cancers by detecting methylation changes in the regulatory region of specific nucleic acid sequences in a sample from a subject.

### BACKGROUND INFORMATION

It has been shown that genetic and epigenetic changes contribute to the development and progression of tumor cells. Epigenetic alterations in promoter methylation and histone acetylation have been associated with cancer-specific expression differences in human malignancies. Methylation has been primarily considered as a mechanism of tumor suppressor gene (TSG) inactivation, and comprehensive whole-genome profiling approaches to promoter hypermethylation have identified multiple novel putative TSGs silenced by promoter hypermethylation.

Understanding the epigenetic changes that lead to cancer progression will help unravel key biologic processes that lead to cancer formation. Thus, there is a need to find molecular markers that will: a) help determine the risk of developing cancer to consider appropriate preventive interventions; b) help detect cancers early when they are amenable to surgical cure; c) help to predict response of a particular therapy (such as paclitaxel); and d) help to determine the overall outcome of a cancer patient.

Briefly, cyclins belong to a highly conserved family, and the members are characterized by a dramatic periodicity in protein abundance through the cell cycle. Cyclins function as regulators of CDK kinases. Different cyclins exhibit distinct expression and degradation patterns which contribute to the temporal coordination of each mitotic event. We previously reported that CCNA1 is frequently methylated in solid tumors including UCC.

Functionally, CCND2 plays different roles in different cancer types. While silencing of CCND2 expression by promoter methylation is associated with cancer progression in some cancer types, over-expression of cyclin D2 correlates with progression and poor prognosis in other tumor types.

### SUMMARY OF THE INVENTION

The present invention is based on the discovery that some genes have regulatory regions, or promoters, that are hypermethylated in cancer. As a result, typically the gene expression is down-regulated and in the case of a tumor suppressor gene, this is a direct cause for cancer cell growth. This discovery is useful for cancer screening, risk-assessment, prognosis, and identification of subjects responsive to a therapeutic regimen. Accordingly, there are provided methods for detecting a cellular proliferative disorder (e.g., urothelial cell carcinoma or UCC) in a subject. The methods of the invention are useful for diagnostic, prognostic (e.g., determining recurrence) as well therapeutic regimen predictions.

In one aspect, promoter methylation of genes, such as cell cycle associated genes, G-protein associated genes, mitogen responsive genes, is a useful tool for analysis of cancer cell growth. Accordingly, a promoter methylation state of genes such as cell cycle associated genes, G-protein associated genes, mitogen responsive genes, is useful for cancer screening, risk-assessment, prognosis, and identification of a subject's responsive to a therapeutic regimen.

In one aspect, ARF, TIMP3, RAR-β2, NID2, CCND2, CCNA1, AIM1, and CALCA are representative members of cell cycle associated genes, G-protein associated genes, mitogen responsive genes.

In one aspect, the promoter methylation state of ARF, TIMP3, RAR-β2, NID2, CCND2, CCNA1, AIM1, and CALCA is useful for cancer screening, risk-assessment, prognosis, and identification of a subject's responsive to a therapeutic regimen. Accordingly, a method is provided for detecting unmethylated cytosine in the promoter of a target gene comprising a) contacting a nucleic acid sample from a subject having or at risk of having a urothelial cell proliferation disorder with a bisulfite preparation, thereby modifying unmethylated cytosine to uracil, b) detecting within the promoter region of one or more of the target genes selected from ARF, TIMP3, RAR-B2, NID2, CCNA1, AIM1, CALCA,CCND2 or any combination thereof, a change in the ratio of cytosine to uracil, wherein, an increase in uracil content of the nucleic acid is indicative of unmethylated cytosine in the promoter of the target gene.

In another embodiment, the invention provides a method for detecting a methylation state of a target gene comprising a) contacting a nucleic acid sample from a subject having or at risk of having a urothelial cell proliferation disorder with a methylation sensitive nucleic acid cleavage composition, thereby generating nucleic acid fragments as cleavage product, b) determining the nucleic acid fragments based on cleavage within the promoter region of a target gene selected from ARF, TIMP3, RAR-2, NID2, CCNA1, AIM1, CALCA, CCND2, or any combination thereof, wherein a change in the ratio of fragmented to unfragmented products due to cleavage within the promoter region of the gene is indicative of the methylation state of the promoter of the target gene.

In one aspect, the invention provides a method for diagnosing or detecting urothelial cancer in a subject having or at risk of developing urothelial cancer or predicting the risk of recurrence of UCC. The method includes determining the methylation state of a gene or a regulatory region of one or more of the ARF, TIMP3, RAR-β2, NID2, CCND2, CCNA1, AIM1, and CALCA genes in a sample from a subject having or suspected of having a urothelial cancer. Such cancers include but are not limited to carcinoma of the bladder, ureter, kidney, or renal pelvis and associated tissues and organs.

A hypermethylated state, as compared to a corresponding normal cell, is indicative of a subject having or at risk of developing urothelial cancer. In one embodiment, the method includes contacting a nucleic acid-containing sample from cells of the subject with an agent that provides a determination of the methylation state of a regulatory region of a ARF, TIMP3, RAR-β2, NID2, CCND2, CCNA1, AIM1, or CALCA gene, wherein, more specifically, the regulatory region of CCND2, CCNA1 or CALCA is hypermethylated in a cell undergoing unregulated cell growth as compared to a corresponding normal cell; and identifying hypermethylation of the regulatory region in the nucleic acid-containing sample, as compared to the same region of the regulatory region in a subject not having urothelial cancer, wherein hypermethylation is indicative of a subject having or at risk of developing urothelial cancer.

In another aspect, the invention provides a method for diagnosing cancer in a subject having or at risk of developing a cell proliferative disorder. The method includes determining the methylation state of the regulatory region of the CCND2, CCNA1 or CALCA gene. A hypermethylated state, as compared to a corresponding normal cell, is indicative of a subject having or at risk of developing a cell proliferative disorder. In one aspect the method includes contacting a nucleic acid-containing sample from cells of the subject with an agent that provides a determination of the methylation state of the CCND2, CCNA1 or CALCA regulatory region (e.g., promoter) of a gene, wherein the regulatory region is hypermethylated in a cell undergoing unregulated cell growth as compared to a corresponding normal cell; and identifying hypermethylation of the regulatory region in the nucleic acid-containing sample, as compared to the same region of the regulatory region in a subject not having urothelial cancer (UC), wherein hypermethylation is indicative of a subject having or at risk of developing a cell proliferative disorder or predictive of recurrence of UC.

In another aspect, the invention provides a method of determining the prognosis of a subject having urothelial cancer. The method includes determining the methylation state of a gene or a regulatory region of the CCND2, CCNA1 or CALCA gene. A hypermethylated state, as compared to a corresponding normal cell in the subject or a subject not having the disorder, is indicative of a poor prognosis.

In another aspect, the invention provides a method of determining the prognosis of a subject having cancer. The method includes determining the methylation state of the regulatory region of CCND2, CCNA1 or CALCA in a nucleic acid sample from the subject. A hypermethylated state, as compared to a corresponding normal cell in the subject or a subject not having the disorder, is indicative of a poor prognosis.

In one aspect, the method of detecting the methylation state of a gene includes obtaining a biological sample (e.g., tissue or fluid sample) from a subject, the sample having nucleic acid of the subject, contacting the nucleic acid in the sample with a nucleic acid modifying composition, such as a bisulfite preparation, wherein contacting the nucleic acid with the bisulfite preparation converts a cytosine to a uracil when the cytosine is present as a member of the cytosine-guanine dinucleotide, i.e., CpG, when the cytosine in a CpG dinucleotide is unmethylated, and, detecting the level of converted uracil to unconverted cytosine as indicative of the methylation state of the region of the DNA under investigation.

Methods of detecting the level of nucleotide base alteration is performed by any known method, such as designing oligonucleotide primers or probes spanning the base alteration, and detecting optimal hybridization with the contacted DNA which was subject to bisulfite conversion.

The primer or probes may be suitably labeled. Detection of hybridization may be performed by southern blot technique, polymerase chain reaction or any related methods known in the art.

Alternatively, the bisulfite contacted DNA may be subjected to nucleotide sequencing of a stretch of polynucleotides in the sample, wherein the stretch contains the CpG sites, and the region interrogated is a region within the promoter of the gene.

In one aspect the method of detecting the methylation state of a gene includes obtaining a biological sample (e.g., tissue or fluid sample) from a subject, the sample having the nucleic acid of the subject, contacting the subject nucleic acid with a methylation sensitive nucleic acid cleavage composition, and determining the nucleotide fragments as cleavage product. A cleavage composition includes compounds such as hydrazine-piperidine, or nucleic acid cleavage enzymes such as restriction endonuclease, wherein the cleavage composition can function based on the methylation state of the cytosine within a CpG site within the cleavage site. Methylation sensitive cleavage of the nucleic acid thereby results in nucleic acid fragments that correspond to whether the CpG within the cleavage or restriction site is methylated or not. Determination of the fragments of nucleic acid is performed by any known method such as identification of fragment length by electrophoresis, mass-spectrophotometry, polymerase chain reaction, or any other methods known in the art.

In one aspect, the regulatory region of the genes may be from about 10 to 1000 bases long. In one aspect, the regulatory region of the genes may be from about 10 to 10,000 or more base pairs long. In one aspect, more than one regions within the regulatory region of the gene are investigated for methylation state determination.

In one aspect, the oligonucleotide primers and probes are from about 5 to 50 nucleotides long. In one aspect, the oligonucleotides are from about 5-100 nucleotides long. In one aspect, the oligonucleotides are from about 5-200 nucleotides long.

In one aspect, the oligonucleotide primer or probes are suitably labeled to enable detection. In some aspects the label is selected from the group consisting of a radioisotope, a bioluminescent compound, a chemiluminescent compound, a fluorescent compound, a metal chelate and an enzyme.

In one aspect of the invention, methylation state may be assessed using real-time methylation specific PCR (QMSP).

The detection of the methylated versus unmethylated cytosine residues within the gene include the identification of the methylation site within the gene, namely the regulatory region within the gene, in this case, the promoter of the target gene.

In another aspect, the invention provides a method of treating cancer or ameliorating symptoms associated with urothelial cancer in a subject in need thereof. The method includes administering to the subject an agent that demethylates a regulatory region of the CCND2, CCNA1 OR CALCA gene. Demethylation of the regulatory region of CCND2, CCNA1 or CALCA that is in a hypermethylated state, as compared to that of a subject not having urothelial cancer, increases expression of the CCND2, CCNA1 or CALCA gene or regulatory region, thereby ameliorating the symptoms associated with urothelial cancer.

In another aspect, the invention provides a method for determining whether a subject is responsive to a particular therapeutic regimen. The method includes determining the methylation state of a gene or a regulatory region of CCND2, CCNA1 or CALCA. A hypermethylated state of the CCND2, CCNA1 or CALCA promoter/regulatory region, as compared with that of a normal subject, is indicative of a subject who may be responsive to the therapeutic regimen. In one embodiment, the therapeutic regimen is administration of one or more chemotherapeutic agents alone or in combination with one or more demethylating agents such as, but not limited to, 5-azacytidine, 5-aza-2-deoxycytidine and zebularine. In another embodiment, the therapeutic regimen is administration of cisplatin and/or paclitaxel.

In another aspect, the invention provides a kit useful for the detection of a methylated CpG-containing nucleic acid in determining the methylation status of CCND2, CCNA1 or CALCA. In one embodiment, the kit includes a carrier element containing one or more containers comprising a first container containing a reagent that modifies unmethylated cytosine and a second container containing primers for amplification of the regulatory region or region of promoter of CCND2, CCNA1 or CALCA, wherein the primers distinguish between modified methylated and unmethylated nucleic acid.

### BRIEF DESCRIPTION OF THE DRAWINGS

FIG. 1 is a scatter plot of quantitative methylation values of the promoters of 8 genes tested in recurrent (R, n=19) and non-recurrent (NR, n=17) primary urothelial cell carcinoma (UCC) samples.

FIG. 2 shows scatter plots showing the extent of methylation in CCNA1, CCND2 and CALCA genes in urine sediment DNA from patients (UCC) and controls (NL).

FIG. 3 shows scatter plots of promoter methylation status of CCNA1, CCND2, and CALCA genes in different grades and stages of UCC.

FIG. 4 shows quantitative reverse transcriptase PCR analysis of CCNA1, CCND2 after 5-Aza-dc and/or TSA treatment to UCC cell lines.

FIGS. 5A and 5B show that transfection and overexpression of CCNA1 inhibits tumor cell growth. A. Cell proliferation analysis by MTT Assay of J82 cells transfected with pCMS-EGF-cyclin Al or mock plasmid. B. Colony formation assay on J82 cells following CCNA1 overexpression.

### DETAILED DESCRIPTION OF THE INVENTION

The present invention is based on the discovery that the CCND2, CCNA1 or CALCA gene promoter or regulatory region is hypermethylated in urothelial cancers. CCND2, CCNA1 or CALCA is a tumor suppressor gene, thus transcriptional down-regulation appears to be associated with cancer. Accordingly, in a first embodiment of the invention, there are provided methods for identifying a cell that exhibits or is predisposed to exhibiting unregulated growth. The method includes determining the methylation state of a regulatory region of CCND2, CCNA1 or CALCA in nucleic acid obtained from a sample from a subject having or suspected of having cancer, wherein the promoter is hypermethylated as compared to a corresponding normal cell not exhibiting unregulated growth, thereby identifying the cell as exhibiting or predisposed to exhibiting unregulated growth.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

It has been shown that genetic changes, which include deletions, amplifications, and mutations in DNA sequence, and epigenetic changes, which refer to heritable changes in the gene expression that occur without changes to the DNA sequence, contribute to the development and progression of tumor cells.

As used herein, the term “hypermethylated” refers to the addition of one or more methyl groups to a cytosine ring in a DNA sequence to form methyl cytosine as compared to a “normal” gene. Such methylations typically only occur on cytosines that precede a guanosine in the DNA sequence, which is commonly known as a CpG dinucleotide. There are CpG-rich regions known as CpG islands which span the 5′ end region (e.g., promoter, untranslated region and exon 1) of many genes and are usually unmethylated in normal cells. The methylation patterns of cancer cells are altered as compared to the corresponding normal cells, undergoing global DNA hypomethylation as well as hypermethylation of CpG islands. Hypomethylation has been hypothesized to contribute to oncogenesis by transcriptional activation of oncogenes and latent transposons, or by chromosome instability. Aberrant promoter hypermethylation and histone modification, leading to transcriptional inactivation and gene silencing, is a common phenomenon in human cancer cells and likely one of the earliest events in carcinogenesis. As such, hypermethylation of CpG islands in gene promoter regions is a frequent mechanism of inactivation of tumor suppressor genes.

As used herein “corresponding normal cells” means cells that are from the same organ and of the same type as the cells being examined, but are known to be free from the disorder being diagnosed or treated. In one aspect, the corresponding normal cells comprise a sample of cells obtained from a healthy individual. Such corresponding normal cells can, but need not be, from an individual that is age-matched to the individual providing the cells being examined. In another aspect, the corresponding normal cells comprise a sample of cells obtained from an otherwise healthy portion of tissue (e.g., bladder, kidney, ureter) of a subject having urothelial cancer.

Accordingly, the present invention is designed to profile methylation alterations on promoter regions of selected genes, e.g., ARF, TIMP3, RAR-132, NID2, AIM1, CCND2, CCNA1 or CALCA, in urothelial tumors with the aim of identifying candidate markers for diagnosis and prognosis of the disease, with sensitivity and specificity necessary to identify subjects with early asymptomatic urothelial cancer, as well as disease monitoring, therapeutic prediction and new targets for therapy.

As used herein, the term “cell proliferative disorder” refers to malignant as well as non-malignant cell populations, which often differ from the surrounding tissue both morphologically and genotypically. In some embodiments, the cell proliferative disorder is a cancer. In particular embodiments the cancer may be a carcinoma. A cancer can include, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, urothelial cancer, testicular cancer, bladder cancer, cervical cancer, and adenomas. In an illustrative example in this invention, the cancer is urothelial cancer.

The nucleic acid-containing sample for use in the invention methods may be virtually any biological sample that contains nucleic acids from the subject. The biological sample can be a tissue sample, which contains 1 to 10,000,000, 1000 to 10,000,000, or 1,000,000 to 10,000,000 somatic cells. However, it is possible to obtain samples that contain smaller numbers of cells, even a single cell in embodiments that utilize an amplification protocol such as PCR. The sample need not contain any intact cells, so long as it contains sufficient material (e.g., protein or genetic material, such as RNA or DNA) to assess methylation status or gene expression levels.

As used herein, the terms “sample” and “biological sample” refer to any sample suitable for the methods provided by the present invention. A sample of cells used in the present method is obtained from tissue samples or bodily fluid from a subject, or tissue obtained by a biopsy procedure (e.g., a needle biopsy) or a surgical procedure. In one embodiment, the biological or tissue sample is drawn from any tissue that is susceptible to cancer. Thus, exemplary samples include, but are not limited to, a tissue sample, a frozen tissue sample, a biopsy specimen, a surgical specimen, a cytological specimen, whole blood, bone marrow, serum, plasma, urine, or ejaculate.

The term “subject” as used herein refers to any individual or patient to which the subject methods are performed. Generally the subject is human, although as will be appreciated by those in the art, the subject may be an animal Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. In addition, the term “subject” may refer to a culture of cells, where the methods of the invention are performed in vitro to assess, for example, efficacy of a therapeutic agent.

As used herein, a “gene” includes its regulatory elements, i.e., its 5′ regulatory elements such as the promoter, enhancer, etc., and the 3′ regulatory elements such as the 3′ untranslated region.

Numerous methods for analyzing methylation status of a gene or regulatory region are known in the art and can be used in the methods of the present invention to identify hypermethylation. As illustrated in the Examples herein, analysis of methylation can be performed by bisulfite genomic sequencing.

Bisulfite ions, for example, sodium bisulfite, convert non-methylated cytosine residues to bisulfite modified cytosine residues. The bisulfite ion treated gene sequence is exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group is removed by exposure to alkaline conditions, resulting in the formation of uracil. The DNA is amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. Therefore, primers or probes are expected to form Watson-Crick base pairing with the respective regions containing the target cytosine-guanine (CpG) dinucleotides, which are sites for cytosine methylation, unless one or more cytosine residues have been converted upon bisulfite treatment. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues are also detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

In another embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Modified products are detected directly, or after a further reaction that creates products that are easily distinguishable. Means, which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali. Other means, which are reliant on specific sequences can be used, including but not limited to hybridization, amplification, sequencing, and ligase chain reaction. Combinations of such techniques can be used as is desired.

In another example, methylation status may be assessed using real-time methylation specific PCR (QMSP). For example, the methylation level of the promoter region of one or more of the target genes can be determined by determining the amplification level of the promoter region of the target gene based on amplification-mediated displacement of one or more probes whose binding sites are located within the amplicon. In general, real-time quantitative methylation specific PCR is based on the continuous monitoring of a progressive fluorogenic PCR by an optical system. Such PCR systems are well-known in the art and usually use two amplification primers and an additional amplicon-specific, fluorogenic hybridization probe that specifically binds to a site within the amplicon. The probe can include one or more fluorescence labeled moieties. For example, the probe can be labeled with two fluorescent dyes: 1) a 6-carboxy-fluorescein (FAM), located at the 5′-end, which serves as reporter, and 2) a 6-carboxy-tetramethyl-rhodamine (TAMRA), located at the 3′-end, which serves as a quencher. When amplification occurs, the 5′-3′ exonuclease activity of the Taq DNA polymerase cleaves the reporter from the probe during the extension phase, thus releasing it from the quencher. The resulting increase in fluorescence emission of the reporter dye is monitored during the PCR process and represents the number of DNA fragments generated.

In other embodiments, hypermethylation can be identified through nucleic acid sequencing after bisulfite treatment to determine whether a uracil or a cytosine is present at a specific location within a gene or regulatory region. If uracil is present after bisulfite treatment, then the nucleotide was unmethylated. Hypermethylation is present when there is a measurable increase in methylation.

In another embodiment, the method for analyzing methylation of the target gene can include amplification using a primer pair specific for methylated residues within the target gene. Thus, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. As such, one primer can selectively bind to a target sequence only when one or more bases of the target sequence is altered by bisulfite treatment, thereby being specific for a methylated target sequence.

Other methods are known in the art for determining methylation status of a target gene, including, but not limited to, array-based methylation analysis (see Gitan et al., Genome Res 12:158-64, 2002) and Southern blot analysis.

Methods using an amplification reaction can utilize a real-time detection amplification procedure. For example, the method can utilize molecular beacon technology (Tyagi S., et al., Nature Biotechnology, 14: 303 (1996)) or TAQMAN™ technology (Holland, P. M., et al., Proc. Natl. Acad. Sci. USA, 88:7276 (1991)).

In addition, methyl light (Trinh, et al. DNA methylation analysis by MethyLight technology, Methods, 25(4):456-62 (2001), incorporated herein in its entirety by reference), Methyl Heavy (Epigenomics, Berlin, Germany), or SNuPE (single nucleotide primer extension) (See e.g., Watson, et al., Genet Res. 75(3):269-74 (2000)) can be used in the methods of the present invention related to identifying altered methylation of the genes or regulatory regions provided herein. Additionally, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

The degree of methylation in the DNA associated with the gene or genes or regulatory regions thereof, may be measured by fluorescent in situ hybridization (FISH) by means of probes that identify and differentiate between genomic DNAs, which exhibit different degrees of DNA methylation. FISH is described in Human chromosomes: principles and techniques (Editors, Ram S. Verma, Arvind Babu Verma, Ram S.) 2nd ed., New York: McGraw-Hill, 1995, and de Capoa A., Di Leandro M., Grappelli C., Menendez F., Poggesi I., Giancotti P., Marotta, M. R., Spano A., Rocchi M., Archidiacono N., Niveleau A. Computer-assisted analysis of methylation status of individual interphase nuclei in human cultured cells. Cytometry. 31:85-92, 1998, which is incorporated herein by reference. In this case, the biological sample will typically be any that contains sufficient whole cells or nuclei to perform short term culture. Usually, the sample will be a tissue sample that contains 10 to 10,000, or, for example, 100 to 10,000, whole somatic cells. However, as indicated above, in one embodiment, the biological sample is a tissue sample which contains from about 1 to 10,000,000, 1000 to 10,000,000, or 1,000,000 to 10,000,000 somatic cells.

In another embodiment, methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used that selectively modify either the methylated or non-methylated form of CpG dinucleotide motifs.

In some embodiments, hypermethylation of the target gene is detected by detecting decreased expression of that gene. Expression of a gene can be assessed using any means known in the art. Typically expression is assessed and compared in test samples and control samples, which may be normal, non-malignant cells. The test samples may contain cancer cells or pre-cancer cells or nucleic acids from the cells. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Moreover, specific proteins can be assessed using any convenient method including, but not limited to, immunoassays and immunocytochemistry. Most such methods will employ antibodies that are specific for the particular protein or protein fragments. The sequences of the mRNA (cDNA) and proteins of the target genes of the present invention are known in the art and publicly available.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon. The microarray analysis process can be divided into two main parts. First is the immobilization of known gene sequences onto glass slides or other solid support followed by hybridization of the fluorescently labelled cDNA (comprising the sequences to be interrogated) to the known genes immobilized on the glass slide (or other solid phase). After hybridization, arrays are scanned using a fluorescent microarray scanner. Analyzing the relative fluorescent intensity of different genes provides a measure of the differences in gene expression. DNA arrays can be generated by immobilizing presynthesized oligonucleotides onto prepared glass slides or other solid surfaces.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC: AT content), and nucleic acid type, i.e., whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, for example, Sambrook et al., supra, 1989).

An example of progressively higher stringency conditions is as follows: 2× SSC/0.1% SDS at about room temperature (hybridization conditions); 0.2× SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, for example, a particular gene of interest, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In yet another aspect, the invention provides methods of determining the prognosis of a subject having urothelial cancer. The method includes determining the methylation state of a regulatory element of a cancer-associated gene. A cancer-associated gene may be a cell-cycle regulating gene, such as members of the cyclin family or other gene identified as having a correlation to cancer. This family is exemplified by CCNA1, CCND2 as examined in the present application. A cancer-associated gene product may be a G protein-coupled receptor or associated protein, for example, CALCA. In the present application, cancer associated genes CCND2, CCNA1 and CALCA are used as exemplary genes whose promoters are evaluated for methylation. A determination of the methylation state of the promoter is indicative of recurrent form of UCC. CCND2, CCNA1 or CALCA regulatory region in a nucleic acid sample from the subject. A comparison of the hypermethylation of the regulatory region, as compared to that of a corresponding normal cell in the subject or a subject not having the disorder, is indicative of a poor prognosis.

In another aspect, the invention provides methods of identifying a cell that exhibits or is predisposed to exhibiting unregulated growth. In another aspect, the invention provides methods of ameliorating symptoms associated with urothelial cancer in a subject in need thereof. The signs or symptoms to be monitored will be characteristic of urothelial cancer and will be well known to the skilled clinician, as will the methods for monitoring the signs and conditions.

As used herein, the terms “administration” or “administering” are defined to include the act of providing a compound or pharmaceutical composition of the invention to a subject in need of treatment. Exemplary forms of administration include, but are not limited to, topical administration, and injections such as, without limitation, intravitreal, intravenous, intramuscular, intra-arterial, intra-thecal, intra-capsular, intra-orbital, intra-cardiac, intra-dermal, intra-peritoneal, trans-tracheal, sub-cutaneous, sub-cuticular, intra-articulare, sub-capsular, sub-arachnoid, intra-spinal and intra-sternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the compound or composition to treat urothelial cancer and/or ameliorate the symptoms associated therewith in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

As used herein, the term “ameliorating” or “treating” means that the clinical signs and/or the symptoms associated with cellular proliferative disorder (e.g., urothelial cancer) are lessened as a result of the actions performed. The signs or symptoms to be monitored will be characteristic of the cellular proliferative disorder (e.g., urothelial cancer) and will be well known to the skilled clinician, as will the methods for monitoring the signs and conditions. Also included in the definition of “ameliorating” or “treating” is the lessening of symptoms associated with urothelial cancer in subjects not yet diagnosed as having the specific cancer. As such, the methods may be used as a means for prophylactic therapy for a subject at risk of having urothelial cancer.

As used herein, the term “demethylating agent” is used to refer to any compound that can inhibit methylation, resulting in the expression of the previously hypermethylated silenced genes. Cytidine analogs such as 5-azacytidine (azacitidine) and 5-aza-2-deoxycytidine (decitabine) are the most commonly used demethylating agents. These compounds work by binding to the enzymes that catalyze the methylation reaction, DNA methyltransferases. Thus, in one embodiment, the demethylating agent is 5-azacytidine, 5-aza-2-deoxycytidine, or zebularine. In another embodiment, the demethylating agent is delivered locally to a tumor site or systemically by targeted drug delivery.

Agents that demethylate the hypermethylated gene or regulatory region of the gene can be contacted with cells in vitro or in vivo for the purpose of restoring normal gene expression to the cell. Once disease is established and a treatment protocol is initiated, the methods of the invention may be repeated on a regular basis to evaluate whether the methylation state of a gene or regulatory region thereof, in the subject begins to approximate that which is observed in a normal subject. Alternatively, or in addition thereto, the methods of the invention may be repeated on a regular basis to evaluate whether the symptoms associated with urothelial cancer have been decreased or ameliorated. The results obtained from successive assays may be used to show the efficacy of treatment over a period ranging from several days to months to years. Accordingly, the invention is also directed to methods for determining whether a subject is responsive to a particular therapeutic regimen by determining an alteration in the methylation state of CCND2, CCNA1 or CALCA regulatory region, as compared to that of a corresponding normal cell in the subject or a subject not having the disorder is indicative of a subject who is responsive to the therapeutic regimen.

In one embodiment, the therapeutic regimen is administration of one or more chemotherapeutic agent. In another embodiment, the therapeutic regimen is administration of one or more chemotherapeutic agents in combination with one or more demethylating agents.

Exemplary chemotherapeutic agents include, but are not limited to, antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin- 14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-a, etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis. Thus, in one embodiment, the therapeutic regimen is administration of paclitaxel.

The materials for use in the methods of the invention are ideally suited for the preparation of a kit. As such, in another aspect, the invention provides a kit for detection of a methylated CpG-containing nucleic acid in determining the methylation status of CCND2, CCNA1 or CALCA promoter region. Such a kit may comprise a carrier device containing one or more containers such as vials, tubes, and the like, each of the containers comprising one of the separate elements to be used in the method. The kit may contain reagents, as described above for differentially modifying methylated and non-methylated cytosine residues. One of the containers may include a probe, which is or can be detectably labeled. Such probe may be a nucleic acid sequence specific for a promoter region associated with CCND2, CCNA1 or CALCA . The kit may also include a container comprising a reporter, such as an enzymatic, fluorescent, or radionucleotide label to identify the detectably labeled oligonucleotide probe.

In certain embodiments, the kit utilizes nucleic acid amplification in detecting the target nucleic acid. In such embodiments, the kit will typically contain both a forward and a reverse primer for each target gene. Such oligonucleotide primers are based upon identification of the flanking regions contiguous with the target nucleotide sequence. Accordingly, the kit may contain primers useful to amplify and screen a promoter region of a CCND2, CCNA1 or CALCA gene.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLES

### Example 1

### Materials and Methods

Tissue and Urine Samples

A total of 36 formalin-fixed paraffin-embedded (FFPE) primary LGPUCC tissues were obtained from patients who underwent therapeutic surgery at The Johns Hopkins Hospital. The demographic and clinical information was obtained from the computerized tumor registry at The Johns Hopkins Healthcare System. Among the 36 LGPUCC samples, 17 were collected from patients who did not recur during any follow-up periods, and the remaining 19 were primary tumor samples that recurred within the follow-up periods after TURBT. We also performed analysis by considering the follow-up periods of 12, 18, and 24 months for recurrence to observe the association with promoter methylation of candidate markers.

To be included in the cohort, an eligible patient had to have a confirmed diagnosis of LGPUCC and a sufficient amount of archived tumor material for DNA extraction.To determine the feasibility of detecting promoter methylation of genes in urine related to LGPUCC recurrence, we tested promoter methylation of 3 genes (CCND2, CCNA1 and CALCA) in the urine sediment of 73 to 148 patients with primary UCC (101 LGPUCC, 24 high grade UCC and 23 unknown grade) and of 56 to 60 healthy subjects without any known neoplastic diseases. Fifty milliliters of voided urine were collected from all cases prior to definite surgery. Urine samples were spun at 3000×g for 10 min and washed twice with phosphate-buffered saline. All samples were stored at −80° C.

Approval for research on human subjects was obtained from The Johns Hopkins University institutional review boards. This study qualified for exemption under the U.S. Department of Health and Human Services policy for protection of human subjects [45 CFR 46.101(b)].

Cell Lines

All of the cell lines (HT1736, T24, J82, UM-UC-3 and SW780) used in this study were cultured accordingly to the recommendations of the American Type Culture Collection (ATCC) (Manassas, VA, USA), from where they were purchased.

DNA Extraction

All original LGPUCC histologic slides were reviewed to reconfirm the diagnosis by a senior urologic pathologist (GN). A representative formalin-fixed paraffin-embedded (FFPE) block that contained sufficient amount of tissue was retrieved for DNA extraction and several 10 micron slides were obtained from each block. The presence of tumor cells was confirmed by staining the first and last slides of the representative block with hematoxylin & eosin. The tumor samples were microdissected to obtain >70% of neoplastic cells. DNA from tumors, cell lines and urine sediments were extracted using the phenol-chloroform extraction protocol followed by ethanol precipitation as described previously [51].

Bisulfite Treatment

DNA extracted from primary tumors, cell lines and urines was subjected to bisulfite treatment, which converts unmethylated cytosine residues to uracil residues, as described previously [52]. EpiTect Bisulfite kit (Cat No. 59104, from QIAGEN Inc. Valencia, Calif.—91355) was used for this conversion, following the manufacturer's instructions.

Quantitative Fluorogenic Methylation Specific PCR (QMSP)

Bisulfite-modified DNA was used as a template for fluorescence-based real-time PCR, as previously described [12] Amplification reactions were carried out in triplicate in a final volume of 20 μL that contained 2 μL of bisulfite-modified DNA; 600 nM concentrations of forward and reverse primers; 200 nM probe; 0.6 U of platinum Taq polymerase (Invitrogen, Frederick, MD); 200 μM concentrations each of dATP, dCTP, dGTP and dTTP; and 6.7 mM MgC12. Primers and probes were designed to specifically amplify the promoter region of ARF, TIMP3, RAR-β2, CCNA1, NID2, AIM1, CALCA, CCND2, and of a reference gene, β-actin; primer and probe sequences and annealing temperatures are provided in Table 1. Amplifications were carried out in 384-well plates in a 7900HT sequence detector (Applied Biosystems, Foster City, Calif.) using the following conditions: 95° C. for 3 minutes, followed by 50 cycles at 95° C. for 15 seconds and 60° C. for 1 minute. Results were analyzed by a sequence detector system (SDS 2.4; Applied Biosystems). Each plate included patient DNA samples, and positive and negative controls. Serial dilutions (90-0.009ng) of in vitro methylated DNA were used to construct a calibration curve for each plate. All samples were within the assay's range of sensitivity and reproducibility based on amplification of internal reference standard (threshold cycle [CT] value for β-actin of 40). The relative level of methylated DNA for each gene in each sample was determined as a ratio of methylation specific PCR-amplified gene to β-actin (reference gene) and then multiplied by 1000 for easier tabulation (average value of triplicates of gene of interest divided by the average value of triplicates of β-actin×1000). The presence or absence of methylation was compared between recurrent and non-recurrent groups using cross-tabulations and χ2 or Fisher's exact tests as appropriate. The cutoff value for each gene was established by maximizing sensitivity and specificity. We determined the empiric cutoff on individual ROC (receiver operating curves) that makes optimal differences between the two groups (maximizing sensitivity and specificity). In our previous study [12], we found that dichotomization and logistic regression essentially produces similar results. Furthermore, considering the small number of sample size, we decided to use empiric cutoffs to see the differences between the two groups.

5-aza-deoxycytidine (5-aza-dc) and Trichostatin A (TSA) Treatment

UCC cells were seeded in 75 cm2 culture flasks at a density of 2×105 and incubated at 37° C. in 5% CO2/95% air overnight. Cells were then treated with 5μM of 5-aza-dc (Sigma Chemical, Sigma, USA) for 5 days. Medium with 5-aza-dc was changed daily.

Additionally, combination treatment with 5-aza-dc and TSA was performed by adding 5μM of 5-aza-dc daily for 5 days and TSA (300 nmol/L; Sigma) was added to the medium for the final 24 hours. Cells were harvested after the last day of treatment (5-aza-dc only and 5 aza-dc+TSA) for RNA extraction and the analysis of gene expression were performed by Quantitative Reverse Transcriptase-PCR (Q-RT-PCR). PBS (phosphate buffered saline) alone was used as a control to exclude non-specific solvent effects on cells. All experiments were run independently twice.

RNA Extraction, cDNA Synthesis and Quantitative Reverse Transcription-PCR (Q-RT-PCR)

RNA was extracted using Qiazol Lysis reagent (Qiagen, Valencia, Calif.) according to the manufacturer's instructions. One microgram of total RNA was used for cDNA conversion using the Quantitect Reverse Transcription Kit (Qiagen, Valencia, Calif.), following manufacturer's protocol.

Q-RT-PCR was performed using the SYBR Green chemistry in a 7900HT Real-Time PCR System (Applied Biosystems, Foster City, CA). The reaction mixture contained 2.6 pl of DEPC-treated water, 5 μl Power SYBR Green PCR Master Mix (Applied Biosystems), and 0.2 μl of gene-specific primers (final concentration, 50 nM each), in a final reaction volume of 10 μl. The RT-PCR primer sequences are available in Supplementary Table 2B. The cycling conditions were as follows: a denaturation step at 95° C. for 3 min, followed by 40 cycles of 95° C. for 15 s, 60° C. for 60 s, and a final step for the generation of a dissociation curve to distinguish between the main RT-PCR product and primer-dimers. Calculations were made with the use of the comparative CT (2_ΔΔCT) method. GAPDH was used as an internal control gene to normalize the reaction for the amount of RNA added to the reverse transcription reactions [53]. Each real-time PCR reaction was performed in triplicates to evaluate the reproducibility of data.

Cellular Viability Assay (MTT Assay)

Cellular proliferation was measured by the thiazolyl blue tetrazolium bromide (MTT) (Sigma-Aldrich) according to the manufacturer's instructions. Briefly, J82 cells were counted and seeded at a density of 1000 cells per well on 96 well plates, in triplicates. The cells were allowed to attach overnight. One plate of cells was seeded in the absence of serum to synchronize growth, while another plate was seeded in the presence of serum (10% FBS). Transfection with the pCMS-EGFP-cyclinA1 and pCMS-EGFP-MOCK (control) vectors (kindly provided by Dr. Jenny L. Persson, Clinical Research Center, Malmo, Sweden) was performed using Fugene HD transfection reagent (Roche). The cell doubling time was calculated during exponential growth phase (0, 24, 48 and 72 hrs). Ten microliters of MTT labeling reagent (5 mg/mL MTT) were added to the culture media without fetal bovine serum (FBS), which was then incubated in the dark for additional 3h at 37° C. This step was followed by cell lysis with the addition of 100pL DMSO. Spectrophotometric readings (A570 nm to A650 nm) were obtained on a Spectra Max 250 96-well plate reader (Molecular Devices). Each assay was carried out in triplicate and each experiment was repeated at least two times.

Transfection and Colony Formation Assay

Colony formation assays were performed in monolayer culture [54]. J82 cells were plated at a density of 2×104 cells/well using 6-well plates, and transfected with 1 μg of either the pCMS-EGFP-cyclinAl or pCMS-EGFP-MOCK (control) vectors using Fugene HD transfection reagent (Roche), according to the manufacturer's protocol. The cells were then detached and plated on 100 mm tissue culture dishes at 24 to 48 hrs post-transfection and simultaneously harvested at 48 hr after transfection to confirm the overexpression of CCNA1 at the mRNA level (Q-RT-PCR) and protein level. Cells were cultured for 2 weeks in medium containing 400 μg/mL of G418 (Cellgro, Manassas, Va.). The cultures were washed twice with phosphate buffered saline (PBS), fixed with 25% acetic acid and 75% methanol at room temperature for 10 minutes, and then stained with 0.1% crystal violet. Colonies were counted and the number of colonies per dish was averaged from three independent experiments that were performed. This colony formation assay was repeated three independent times.

Statistical Analysis

The presence or absence of methylation was compared between the groups (recurrent and non-recurrent UCC; and urine of UCC cases and controls) using cross-tabulations and ×2 or Fisher's exact tests as appropriate. Student t-test was used to compare the averages of duplicates or triplicates among the re-expression experiments, cell viability and colony formation assays.

### Example 2

### Detection and Quantitation of DNA Methylation by PCR

Methylation at cytosine residues is effectively detected and quantitated by quantitative fluorogenic methylation specific PCR (QMSP). The assay is performed as described (see Maldonado, L. et al., Oncotarget, 2014, 5(14): 5218-5233). Briefly, DNA is extracted from cells isolated from a biological sample by phenol-chloroform extraction protocol followed by ethanol precipitation as described previously (Hogue, M. et al., J. Clin Oncol. 2005, 23(27): 6569-6575). Next it is subjected to bisulfite treatment, which converts unmethylated cytosine residues to uracil residues, as specified in the manufacturer's instructions (EpiTect Bisulfite kit, Qiagen), as previously described (Herman J G. et al., Proc Natl Acad Sci USA. 1996, 93 (18): 9821-9826). Bisulfite-modified DNA was used for fluorescence-based real-time PCR, as previously described (Hogue, M. et al., J Natl Cancer Inst. 2006, 98(14): 996-1004) Amplification reactions are carried out in triplicate in a 20 μl reaction volume, containing 2 μl bisulfite modified DNA; 600 nM concentration of forward and reverse primes; 200 nM probe; 0.6 U of platinum Taq polymerase (Invitrogen, Frederick, MD); 200 pM concentrations each of dATP, dCTP, dGTP and dTTP; and 6.7 mM MgCl₂. Primers and probes are designed to specifically amplify the promoter region of ARF, TIMP3, RAR-β2, CCNA1, NID2, AIM1, CALCA, CCND2 and a reference gene, β-actin. Table 1 provides the primer probe sequences and annealing temperatures.

Amplifications are carried out in 384-well plates in a 7900HT sequence detector (Applied Biosystems), using the following conditions: 95 oC for 3 minutes, followed by 50 cycles at 95 oC for 15 seconds and 60 oC for 1 minute. Results were analyzed by a sequence detector system (SDS 2.4; Applied Biosystems). Each plate includes patient DNA samples, and positive and negative controls. Serial dilutions (90-0.009 ng) of in vitro methylated DNA are used to construct a standard calibration curve for each plate. All the samples were within the assay's range of sensitivity and reproducibility based on amplification of internal reference standard (threshold cycle, CT value for β-actin of 40).

The relative level of methylated DNA for each gene in each sample are determined as a ratio of methylation specific PCR amplified target gene to amplified 13-actin reference gene. For ease of representation, the ratio of the average of triplicate CT readings for each target gene, and the average of triplicate readings for the β-actin gene is then multiplied by 1000 and tabulated.

The presence and absence of methylation was compared between recurrent and non-recurrent groups using cross-tabulations and Chi-Square or Fisher's exact tests as appropriate. The cutoff value for each gene was established by maximizing sensitivity and specificity.

TABLE 1
A. Primers and probes sequences and annealing
temperatures (T° C.) used for QMSP
(SEQ ID NO: 1-27)

| | Forward | Probe 5′-3′ | Reverse | |
|---|---|---|---|---|
| | 5′-3′ | (6-FAM-5′-3′- | 5′-3′ | |
| Gene | (primer) | 6-TAMRA) | (primer) | T° C. |
| βActin | TGGTGATGG | ACCACCACCCA | AACCAATAA | 60 |
| | AGGAGGTT | ACACACAATA | AACCTACTC | |
| | TAGTAAGT | ACAAACACA | CTCCCTTAA | |
| A1M1 | CGCGGGTAT | GGGAGCGTT | CCGACCCAC | 60 |
| | TGGATGTT | GCGGATTA | CTATACGA | |
| | AGT | TTCGTAG | AAA | |
| ARF | ACGGGCGTT | CGACTCTAA | CCGAACCTC | 60 |
| | TTCGGTAGT | ACCCTACGC | CAAAATCT | |
| | T | ACGCGAAA | CGA | |
| CALCA | GTTTTGGAA | ATTCCGCCAA | TTCCCGCCG | 60 |
| | GTATGAGG | TACACAACAA | CTATAAAT | |
| | GTGACG | CCAATAAACG | CG | |
| CCN | TCGCGGCGA | CGTTATGGC | CCGACCGCG | 60 |
| A1 | GTTTATTCG | GATGCGGTT | ACAAACG | |
| | | TCGG | | |
| CCND2 | TTTGATTTA | AATCCGCCA | ACTTTCTCC | 60 |
| | AGGATGCGT | ACACGATCG | CTAAAAACC | |
| | TAGAGTACG | ACCCTA | GACTACG | |
| NID2 | GCGGTTTTT | ACGCCGCTA | CTACGAAA | 60 |
| | AAGGAGTT | CCCCAAACC | TTCCCTTT | |
| | TTATTTTC | TTACGA | ACGCT | |
| RARβ2 | GGGATTAGA | TGTCGAGAA | TACCCCGA | 60 |
| | ATTTTTTAT | CGCGAGCGA | CGATACCC | |
| | GCGAGTTGT | TTCG | AAAC | |
| T1MP3 | GCGTCGGAG | AACTCGCTC | CTCTCCAA | 62 |
| | GTTAAGG | GCCCGCCGA | AATTACCG | |
| | TTGTT | A | TACGCG | |

### Example 3

### Promoter Methylation Detection in UCC Tumor Tissue Samples

DNA is extracted from formalin fixed paraffin embedded (FFPE) block containing tumor tissue. A representative FFPE block, reviewed and confirmed to contain the pathologic sample is sectioned to obtain multiple 10 micron slides, several of which are used for microdissection to obtain portions containing greater than 70% of neoplastic cells. The first and last slides of the representative block are stained with hematoxillin and eosin.

By a candidate gene approach, promoter methylation of 8 genes (ARF, TIMP3, RAR-β2, NID2, CCNA1, AIM1, CALCA and CCND2) were analyzed by quantitative methylation specific PCR (QMSP) in the DNA of 17 non-recurrent and 19 recurrent noninvasive low grade papillary urothelial cell carcinoma archival tissues. A total of 36 FFPE primary low-grade papillary urothelial cell carcinoma (LGPUCC) tissue samples were obtained from patients who underwent therapeutic surgery. Among them 17 samples were from patients who did not show a recurrence while 19 were from samples that recurred after trans-urethral resection of bladder tumor (TURBT) within the follow up period of up to 24 months. A detailed summary of the LGPUCC samples with their clinic-pathological

parameters is given in Table 2B, below.

TABLE 2A
Promoter methylation frequency in tissues and urines.
A. Promoter methylation frequency for the 8 genes analyzed in the
primary LGPUCC samples (non-recurrent versus recurrent)

| | Methylation positive % (number of | | |
|---|---|---|---|
| | methylation positive/number of total cases) | | Fisher's |
| | Non-recurrent | | exact test |
| GENE | tumors | Recurrent tumors | p-value |
| CCND2 | 2/17 (11.7%) | 10/19 (52.6%) | 0.014* |
| CCNA1 | 4/17 (23.5%) | 11/19 (57.9%) | 0.048* |
| CALCA | 4/17 (23.5%) | 10/19 (52.6%) | 0.097 |
| AIM1 | 8/17 (47%) | 14/19 (73.9%) | 0.171 |
| NID2 | 3/17 (17.6%) | 13/19 (68.4%) | 0.003* |
| ARF | 2/17 (11.7%) | 0/19 (0%) | 0.216 |
| TIMP3 | 10/17 (58.8%) | 4/19 (21%) | 0.039* |
| RARβ2 | 5/17 (29.4%) | 3/19 (15.8%) | 0.434 |

B. Promoter methylation of CCND2, CCNA1 and CALCA in urine of UCC
patients and controls, and its association with clinicopathological parameters
I. Promoter methylation frequency in urine from controls and UCC cases

| | Methylation positive % (number of | | |
|---|---|---|---|
| | methylation positive/number of total cases) | | Fisher's |
| | Normal urines | | exact test |
| GENE | (controls) | UCC urines | p-value |
| CCND2 | 0/56 (0%) | 38/148 (25.6%) | <0.0001* |
| CCNA1 | 10/60 (16.6%) | 50/73 (68.4%) | <0.0001* |
| CALCA | 16/56 (28.5%) | 94/148 (63.5%) | <0.0001* |

II. Association of Promoter methylation determined in
urine with grade and stage of UCC

| | Methylation positive % (number of | | Fisher's |
|---|---|---|---|
| | methylation positive/number of total cases) | | exact test |
| GENE | LGUCC | HGUCC | p-value |
| CCND2 | 35/101 (34.6%) | 3/24 (12.5%) | 0.047* |
| CCNA1 | 35/52 (67.3%) | 7/14 (50%) | 0.348 |
| CALCA | 76/101 (75.2%) | 8/24 (33.3%) | 0.0002* |
| Non-invasive stage (Stage 1) | | Invasive stages (Stage 2, 3) | |
| CCND2 | 3/32 (9.3%) | 35/92 (38.1%) | 0.002* |
| CCNA1 | 9/16 (56.3%) | 34/49 (69.4%) | 0.372 |
| CALCA | 17/32 (53.1%) | 67/92 (72.8%) | 0.049* |

* p values < 0.05 were considered statistically significant

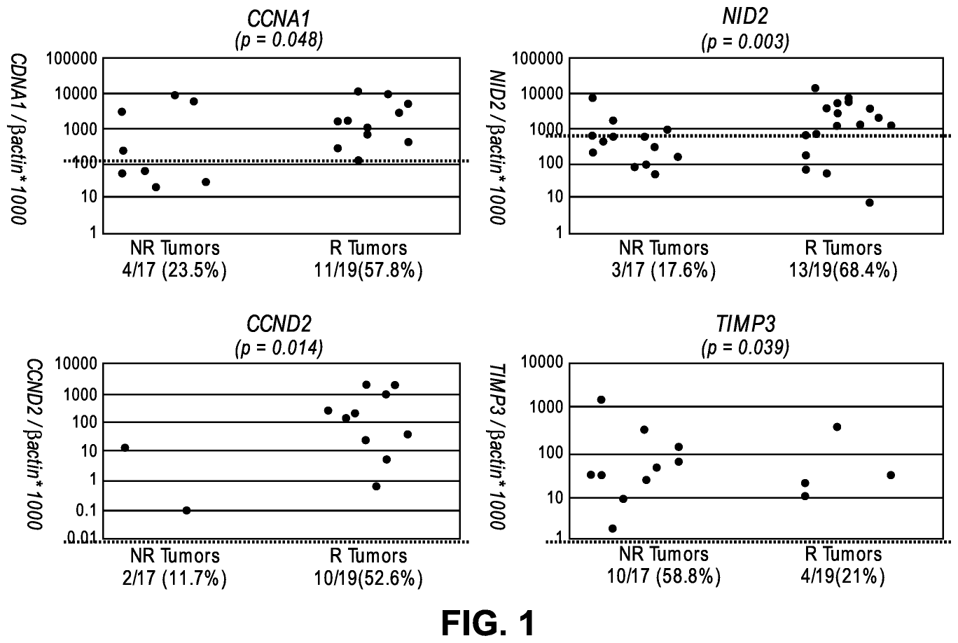

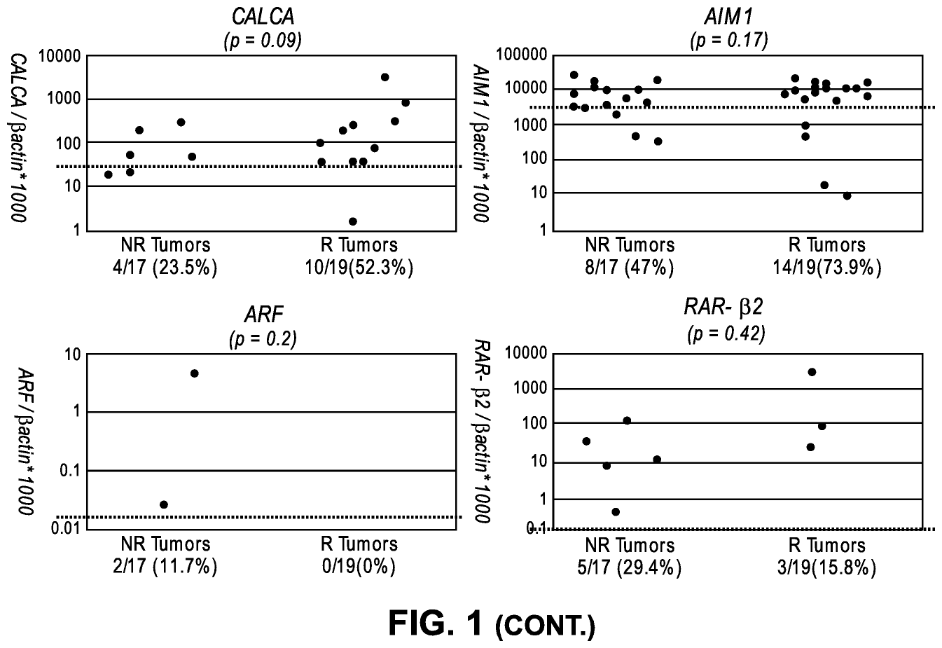

Analysis of promoter methylation of 8 genes (ARF, TIMP3, RAR-β2, NID2, CCNA1, AIM1, CALCA and CCND2) in DNA from primary non-recurrent and recurrent LGPUCC tissues was done. By establishing empiric cutoff values, CCND2, CCNA1, NID2, and CALCA showed a significantly higher frequency of methylation in recurrent than in non-recurrent LGPUCC (Table 2A). The methylation frequency of an individual gene in recurrent and non-recurrent LGPUCC respectively was: CCND2 10/19 (52.6%) vs. 2/17 (11.7%) (p=0.014); CCNA1 11/19 (57.9%) vs. 4/17 (23.5%) (p=0.048); NID2 13/19 (68.4%) vs. 3/17 (17.6%) (p=0.003); and CALCA 10/19 (52.6%) vs. 4/17 (23.5%) (p=0.097). Scatter plots of all the 8 genes tested are shown in FIG. 1. Scatter plots of quantitative methylation values of all the 8 genes tested in recurrent (R, n=19) and non-recurrent (NR, n=17) primary urothelial cell carcinoma (UCC) samples. Calculation of the gene of interest/β-actin ratios was based on the fluorescence emission intensity values for both the gene of interest and β-actin obtained by quantitative real-time PCR analysis. The obtained ratios were multiplied by 1,000 for easier tabulation. Zero values cannot be plotted correctly on a log scale.

TABLE 2B
Demographic and clinicopathological
data of primaryLGUCC samples*

| Age at diagnosis (years) | |
|---|---|
| Median | 66.4 |
| Range | 31-89 |
| Recurrence | |
| Recurrent | 19 (52.7%) |
| Non-recurrent | 17 (47.2%) |
| Race | |
| Caucasian | 31 (86.1%) |
| African-american | 2 (5.6%) |
| Unknown | 3 (8.3%) |
| Gender | |
| Male | 30 (83%) |
| Female | 6 (17%) |
| Smoking | |
| Smoker | 22 (61.1%) |
| Non-smoker | 10 (27.8%) |
| Unknown | 4 (11.1%) |

*All patients were diagnosed with Low Grade Papillary Urothelial Cell Carcinoma

### Example 4

### Promoter Methylation Detection in Patient Urine Samples

The methylation status of CCND2, CCNA1, and CALCA genes is detectable through a simple and low-cost method using urine samples. This panel of genes can be used for early detection of recurrence of non-invasive urothelial bladder cancer, which has high risk of recurrence requiring frequent, invasive, and expensive surveillance. Unlike the current standard urine cytology, this DNA based method does not require a highly trained cytopathologist for interpretation and can detect recurrence with higher sensitivity. This method can be performed in a non-invasive way using a voided urine sample and at a much lower cost compared to the standard cystoscopy.

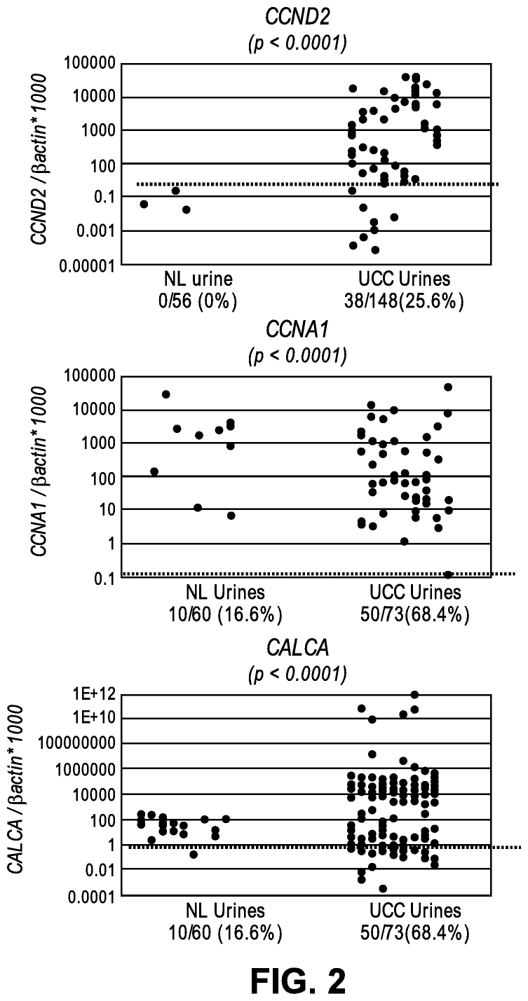

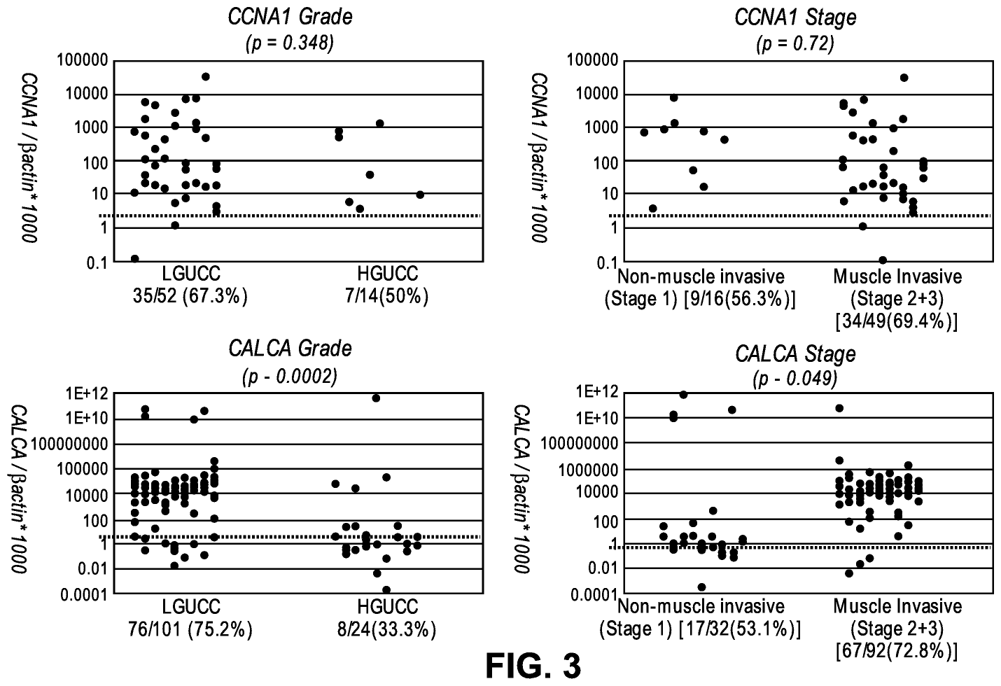

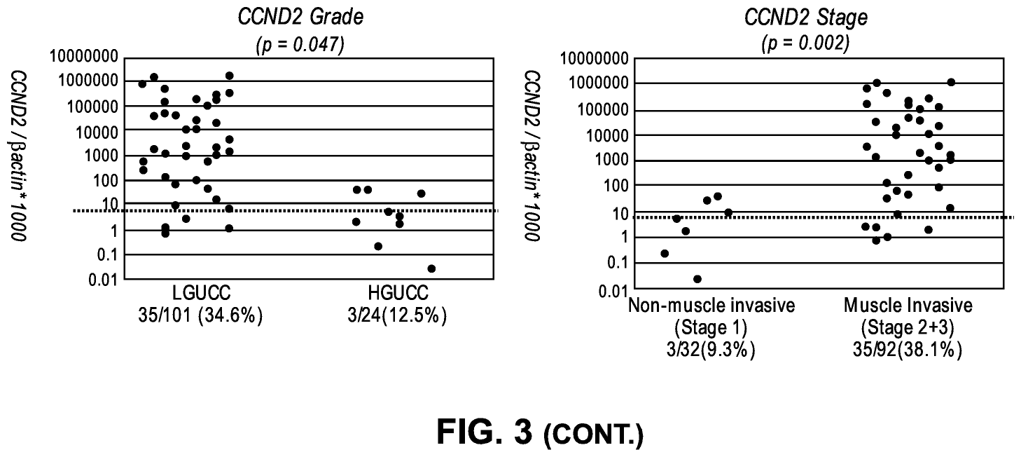

Detection of promoter methylation of CCND2, CCNA1, and CALCA genes in urine samples is used for early detection and monitoring of low grade papillary urothelial cell carcinoma patients. 50 μl of voided urine were collected from nearly 148 samples with LGPUCC and high grade UCC prior to definite surgery, and 56 healthy controls. Urine samples were spun at 3000×g for 10 minutes and washed twice with phosphate-buffered saline. All urine sediment samples were stored at −80° C. until DNA extraction. By analyzing the methylation status of candidate genes in urine samples of noninvasive low grade urothelial cell carcinoma patients through QMSP, CCND2, CCNA1, and CALCA genes were identified as highly methylated in the promoter regions in recurrent cohort compared to non-recurrent control. FIG. 2 demonstrates higher promoter methylation of these genes. The frequency of CCND2, CCNA1, and CALCA was significantly higher (p<0.0001) in urine of urothelial cell carcinoma cases [38/148 (25.6%), 50/73 (68.4%) and 94/148 (63.5%) respectively] than controls [0/56 (0%), 10/60 (16.6%) and 16/56 (28.5%), respectively)]. FIGS. 2 and 3 show Scatter plots showing the extent of methylation in CCNA1, CCND2 and CALCA genes in urine sediments; FIG. 2. Methylation levels of CCNA1, CCND2 and CALCA genes in urine sediment DNA of UCC patients (148 for CCND2, 73 for CCNA1 and 148 for CALCA) and no known neoplastic disease subjects (56 for CCND2, 60 for CCNA1 and 56 for CALCA). NL=Normal Controls, UCC=Urothelial Cell Carcinoma. FIG. 3: Scatter plots showing promoter methylation status of CCNA1, CCND2, and CALCA genes in different grade and stages of UCC. A high percentage of LGUCC can be determined by each of the gene tested. Interestingly, 83% (25/30) of cytology negative LGPUCC cases were positive for one or more of the three methylation markers tested in urine. Out of 101 LGUCC cases, cytology data was available for 70 cases. Detailed information on the methylation and cytology test results of these 70 cases is available in Table 3.

Table 3 provides the clinicopathological and molecular characteristics of urine samples from LGUCC patients tested. Most importantly, we found at least one of the 3 markers were methylated positive in 25 out of 30 (83%) cytology negative low grade papillary urothelial cell carcinoma cases. The study clearly demonstrates that the methylation status of the promoter could be detected in the urine samples, and is a sensitive indicator of early stage of the disease.

TABLE 3
Clinicopathological and molecular charac-
teristics of urine samples from
LGUCC patients tested

| | | | Re- | | CC | CC | | Any |
|---|---|---|---|---|---|---|---|---|
| | Cytol- | Cysto- | cur- | | NA | ND | | Posi- |
| ID | ogy | scopy | rence | Grade | 1 | 2 | CALCA | tive |
| 1 | + | + | − | LGUCC | NA | — | — | — |
| 2 | + | + | — | LGUCC | NA | — | — | — |
| 3 | + | + | — | LGUCC | NA | — | — | — |
| 4 | + | — | — | LGUCC | NA | — | — | — |
| 5 | + | NA | — | LGUCC | NA | — | + | + |
| 6 | + | — | — | LGUCC | NA | — | + | + |
| 7 | + | + | + | LGUCC | NA | — | + | + |
| 8 | + | + | — | LGUCC | NA | — | — | — |
| 9 | + | NA | — | LGUCC | NA | — | — | — |
| 10 | + | — | NA | LGUCC | NA | + | + | + |
| 11 | + | + | — | LGUCC | NA | + | + | + |
| 12 | + | + | — | LGUCC | NA | + | + | + |
| 13 | + | NA | — | LGUCC | NA | — | + | + |
| 14 | + | + | NA | LGUCC | NA | — | — | — |
| 15 | + | + | + | LGUCC | — | — | + | + |
| 16 | + | + | + | LGUCC | NA | — | — | — |
| 17 | + | + | — | LGUCC | — | + | + | + |
| 18 | + | + | — | LGUCC | + | + | + | + |
| 19 | + | + | — | LGUCC | + | — | — | + |
| 20 | + | + | — | LGUCC | + | + | + | + |
| 21 | + | — | — | LGUCC | — | + | + | + |
| 22 | + | + | — | LGUCC | + | — | — | + |
| 23 | + | + | — | LGUCC | + | — | + | + |
| 24 | + | + | — | LGUCC | — | — | + | + |
| 25 | + | + | — | LGUCC | — | + | + | + |
| 26 | + | + | + | LGUCC | — | — | + | + |
| 27 | + | + | — | LGUCC | — | — | — | — |
| 28 | + | + | — | LGUCC | + | — | + | + |
| 29 | + | + | — | LGUCC | + | + | + | + |
| 30 | + | + | + | LGUCC | NA | — | — | — |
| 31 | + | + | — | LGUCC | + | — | + | + |
| 32 | + | + | — | LGUCC | + | — | + | + |
| 33 | + | — | NA | LGUCC | + | + | + | + |
| 34 | + | + | — | LGUCC | — | — | + | + |
| 35 | + | + | — | LGUCC | — | + | + | + |
| 36 | + | + | — | LGUCC | — | + | + | + |
| 37 | + | + | — | LGUCC | — | + | — | + |
| 38 | + | + | — | LGUCC | — | + | + | + |
| 39 | + | + | — | LGUCC | — | — | + | + |
| 40 | + | + | — | LGUCC | — | + | + | + |
| 41* | — | — | — | LGUCC | — | — | + | + |
| 42* | — | + | + | LGUCC | — | — | + | + |
| 43 | — | + | — | LGUCC | — | — | — | — |
| 44* | — | + | — | LGUCC | — | — | + | + |
| 45* | — | + | + | LGUCC | — | — | + | + |
| 46* | — | + | + | LGUCC | — | — | + | + |
| 47* | — | + | NA | LGUCC | — | — | + | + |
| 48* | — | — | — | LGUCC | — | — | + | + |
| 49* | — | + | — | LGUCC | + | — | + | + |
| 50 | — | + | + | LGUCC | — | — | — | — |
| 51 | — | + | — | LGUCC | — | — | — | — |
| 52* | — | + | — | LGUCC | + | — | + | + |
| 53* | — | + | — | LGUCC | + | — | — | + |
| 54* | — | — | + | LGUCC | + | — | + | + |
| 55* | — | + | + | LGUCC | + | + | + | + |
| 56* | — | + | + | LGUCC | + | — | + | + |
| 57 | — | + | — | LGUCC | — | — | — | — |
| 58* | — | + | — | LGUCC | + | + | + | + |
| 59 | — | + | — | LGUCC | — | — | — | — |
| 60* | — | + | — | LGUCC | + | + | + | + |
| 61* | — | + | — | LGUCC | + | — | + | + |
| 62* | — | + | — | LGUCC | + | — | + | + |
| 63* | — | + | — | LGUCC | + | — | + | + |
| 64* | — | + | — | LGUCC | — | — | + | + |
| 65* | — | + | — | LGUCC | — | — | + | + |
| 66* | — | + | — | LGUCC | + | + | + | + |
| 67* | — | + | — | LGUCC | NA | — | + | + |
| 68* | — | + | — | LGUCC | NA | + | + | + |
| 69* | — | + | — | LGUCC | NA | + | + | + |
| 70* | — | + | — | LGUCC | NA | + | + | + |

*Cytology negative but promoter methylation positive
NA, sample was not available for testing

### Example 5

### Effect of Epigenetic Drug on Expression of CCNA1 and CCND2

Quantitative reverse transcriptase PCR was performed to determine whether promoter methylation of CCNA1 and CCND2 inversely correlated with their expression. Briefly, UCC cells were seeded in 75 cm² cell culture flasks at the density of 2×10⁵ cells per ml, and incubated overnight at 37° C., with 5% CO₂. Cells were treated with DNA methylation inhibitor 5 μM of 5-aza-dc (Sigma Chemicals), for 5 days. Medium with 5-aza-dc was changed daily. In certain cases the cells were additionally treated with 300 nmol/L for an additional 24 hours. CCNA1 and CCND2 expressions were tested from 1 μg total RNA from the cells by quantitative reverse transcription-PCR (Qiagen), using SYBR Green chemistry in a 7900HT Real-time PCR system (Applied Biosystems). The reaction mixture contained 2.6 μl of DEPC-treated water, 5 μl SYBR Green PCR Master Mix (Applied Biosystems), and 0.2 μl of gene specific primers, final concentration of 50 nM each. RT-PCR primers are provided in Table 4. The PCR was run for 40 cycles after a 3 minute- initial denaturation step at 95° C., at 95° C., 15 seconds, 60° C., 60 seconds each, with a final step of generation of dissociation curve for validation of an unique DNA amplification product.

Two UCC cell lines (SW780 and J82) showed re-expression of CCNA1 after 5-aza-dc treatment (p<0.001) and after combination treatment (p<0.05 in J82 and p<0.001 in SW780) (FIG. 3A). CCND2 showed a similar pattern of re-expression with 5-aza-dc treatment (UMUC- 3, J82 and T24) and after combination treatment (UMUC-3, J82, T24 and SW780). CCND2 expression was down-regulated only in the HT1376 cell line after treatment with 5-aza-dc and trichostatin-A (FIG. 3B). To determine whether promoter methylation of CCNA1 and CCND2 are inversely correlated with expression, we performed QMSP assay for CCNA1 and CCND2. Among the 5 UCC cell lines, promoter methylation of CCNAI is inversely correlated with expression in J82 and SW780 (data not shown). Similarly, for CCND2, we observed that promoter methylation is inversely correlated with expression in J82, SW780 and T24 cell lines (data not shown). These findings suggest that both DNA methylation and histone deacetylation play a role in CCND2 and CCNA1 genes silencing.

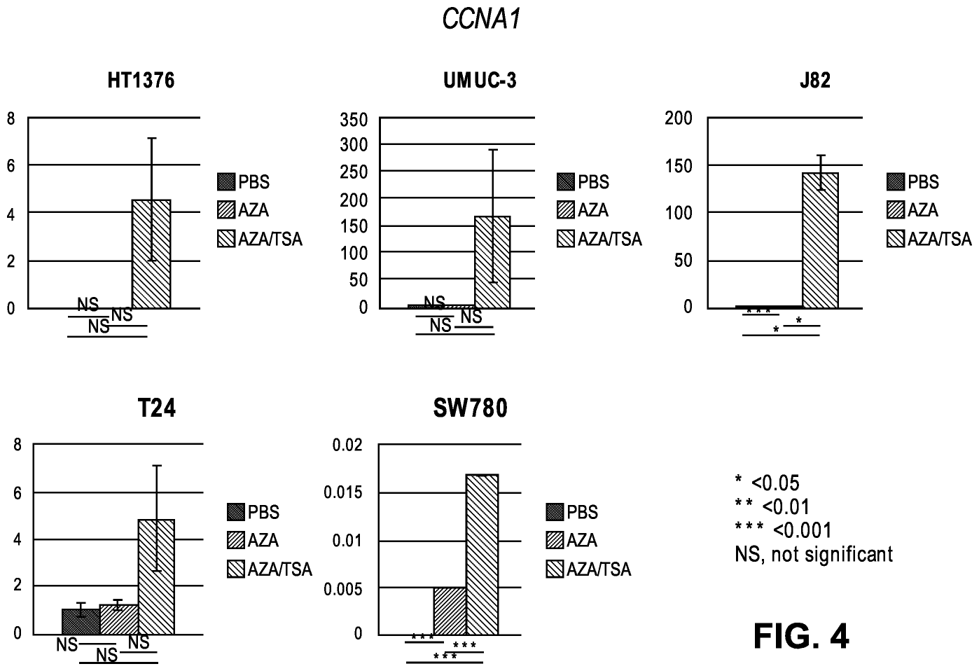

FIG. 4 demonstrates that UCC cell lines treated with methylation inhibitor 5-aza alone or in combination with TSA, which is a histone deacetylase inhibitor, restores the expression of CCNA1 and CCND2 from the 8 gene panel. FIG. 4 shows re-expression of CCNA1 and CCND2 after 5-aza-dc (AZA) and/or TSA treatment of urothelial cancer (UCC) cell lines analyzed by real-time RT-PCR. A. Reactivation of CCNA1 was observed in SW780 and J82 UCC cell lines after 5-aza-dc treatment (p<0.001), while robust overexpression of CCNA1 was observed after combination treatment (p<0.05). B. Reactivation of CCND2 was observed in UMUC-3, J82 and T24 UCC cell lines after 5-aza-dc treatment (p<0.05). When using combination treatment with 5-aza and TSA, an increased expression was observed in UMUC-3, J82, T24 and SW780 cell lines (p<0.05). In HT1376 cell line, overexpression was observed after 5-aza-dc treatment only (not significant), however, CCND2 expression noticeably decreased after combination treatment of 5-aza-dc and TSA treatment. PBS was used as treatment control. PBS, phosphate buffered saline; AZA, 5-aza-dc; TSA, trichostatin-A; AZA/TSA, combination treatment with 5-aza-dc and trichostatin-A; NS, not significant; *, p<0.05; **, p<0.01; ***, p<0.0001. t-student test p values.

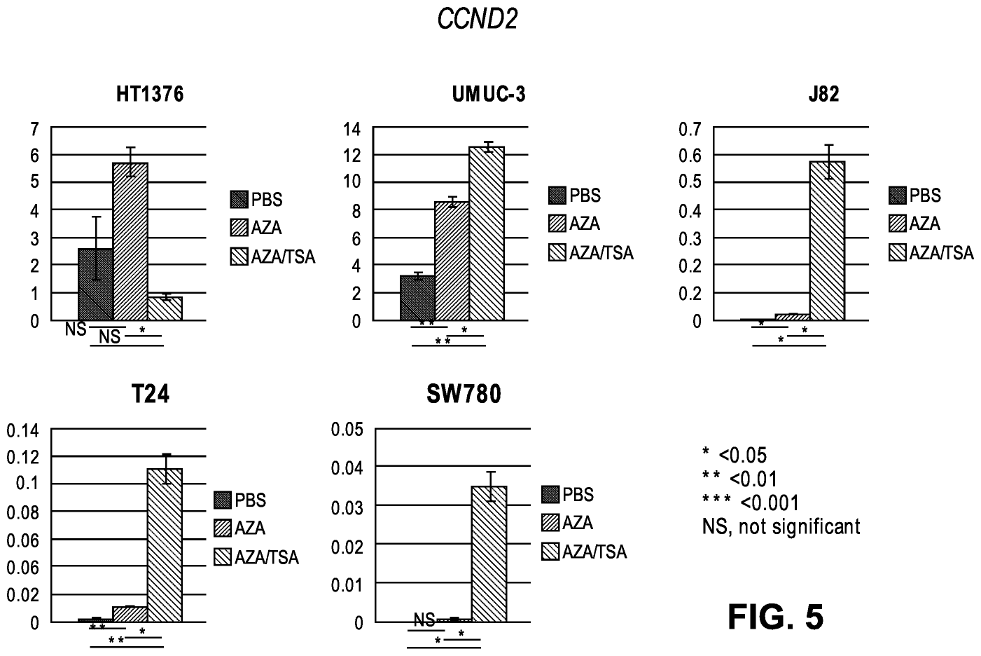

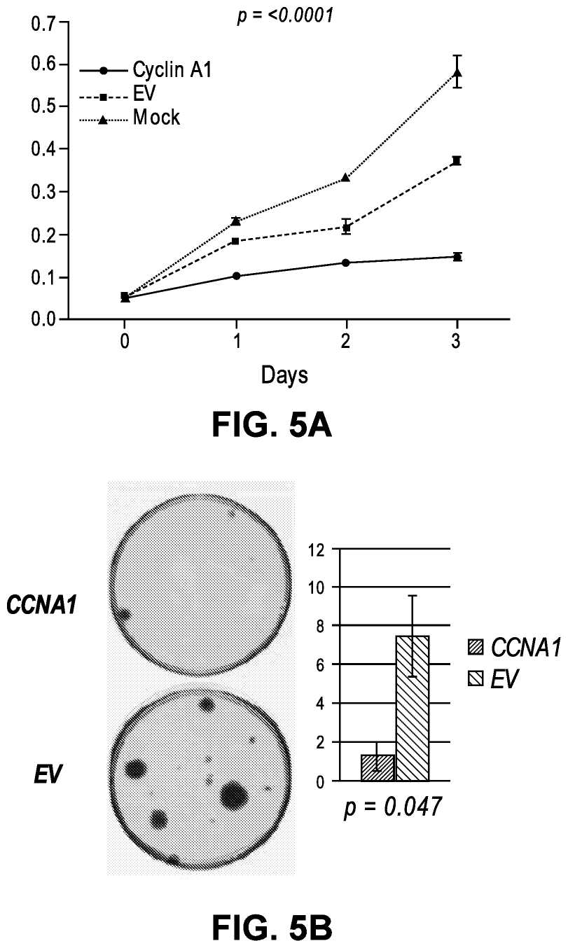

These findings as a proof of principle indeed showed that CCND2 and CCNA1 can be re-expressed with the treatment of epigenetic drugs. Additionally, the implication of the suppression of CCNA1 expression by promoter methylation in UCC, and the ability to restore its expression by methylation inhibitor drug, is further exemplified by overexpression of CCNA1 in UCC cell line J82. 2×104 J82 cells were transfected with 1□g of pCMS-EGFP-cyclin A or mock control plasmids using Fugene HD transfection reagent (Roche). CCNA1 overexpression significantly inhibited the growth of J82 cells in culture, measured by MTT assay (Sigma Aldrich), and markedly reduced the colony-forming ability (FIG. 5). FIGS. 4A and 5B show ectopic expression of CCNA1 inhibits tumor cell growth. A. The MTT assay was performed in a J82 cell line transiently transfected with pCMS-EGFP-cyclinAl and empty pCMS-EGFP plasmid (control). Forceful expression of CCNA1 significantly decreased the viable cells in comparison with empty vector (EV) control and cells without any transfection (Mock) (p=<0.0001) B. The effect of ectopic CCNA1-expression on bladder carcinoma cell clonogenicity was investigated by colony formation assay. J82 cells were transfected with pCMS-EGFP-cyclinAl and empty pCMS-EGFP plasmid (control). Left panel, images of the colony formation assays. Right panel, Bar graph representing the number of colonies observed (larger than 2 mm). Significantly fewer numbers of colonies were observed after over expressing CCNA1 containing vector in J82 cells (p=<0.047).

To evaluate the effect of CCNA1 on the growth of UCC cell lines, CCNA1 was forcefully expressed in J82 cell line. Verification of CCNA1 overexpression was done by Q-RT-PCR and immunoblotting analysis 48h after transfection (data not shown). As shown in FIG. 5A, forced expression of CCNA1 significantly inhibited growth of J82 cells in culture (p=<0.0001), where cell growth inhibition is mediated in a time-dependent manner To assess long-term growth, colony focus assays were performed after treatment of CCNA1 transfected cells with the plasmid selection marker G418 for 2 weeks. CCNA1 showed potent tumor suppressive activity by markedly reducing the colony-forming ability of the cells as shown in FIG. 5B.

TABLE 4
Primers sequences and annealing temperatures
(T° C.) used for Quantitative real-time RT-PCR.

| | Forward 5′-3′ | Reverse 5′-3′ | |
|---|---|---|---|
| Gene | (primer) | (primer) | T° C. |
| GAPDH | CGTCTTCACC | CGGCCATCAC | 58 |
| | ACCATGGAGA | GCCACAGTTT | |
| CCNA1 | CTCCTGTCTG | TCAGGTGTTAT | 58 |
| | GTGGGAGGA | TCTGGATCAG | |
| CCND2 | CGCAAGCATG | CCACCGTCG | 58 |
| | CTCAGACCTT | ATGATCGCA | |

Summary: The main goal of this study was to evaluate whether the status of promoter methylation of a candidate gene or gene-panel was different among LGPUCC that recurred and those that did not. For further monitoring of patients after TURBT of LGPUCC, a non-invasive screening test is essential in order to avoid invasive and costly procedures such as cystoscopy. To this end, we evaluated the feasibility of a set of genes that predicts recurrence in primary LGPUCC for the non-invasive detection of UCC in urine sediments. To elucidate the biologic relationship of CCNA1 silencing in the context of UCC, we performed different in vitro assays and our data is consistent with our findings in human primary LGPUCC that CCNA1 is a potential tumor suppressor gene.

We analyzed promoter methylation of 8 genes (ARF, TIMP3, RAR-β2, NID2, CCNA1, AIM1, CALCA and CCND2) in the recurrent and non-recurrent LGPUCC and observed that the methylation frequencies of 3 genes (NID2, CCNA1, and CCND2) were significantly higher in recurrent LGPUCC. The frequency of promoter methylation of CALCA was borderline significant (p=0.09). We had previously shown a UCC specific methylation pattern for CCND2, CCNA1 and CALCA [11]. In the latter study, we analyzed 93 UCC samples and 26 normal uro-epithelium samples and observed 57% of methylation in CCNA1 in tumors while no methylation was observed in controls, 57% in CCND2 in tumors while 19% in normals, and 65% in CALCA with 15% in normal uro-epithelium [11]. AIM1, a gene without a clear functional data, showed a UCC specific pattern (over 70% in UCC) in our previous study [11], however, although we found high frequency of methylation in the tested primary LGPUCC samples in this study [22/36(61%)], AIM1 was not differentially methylated among recurrent and non-recurrent LGPUCC. This could be due to small sample size in that study or AIM1 inactivation may be related to both initiation and progression of UCC. Ulazzi et al., were the first group to demonstrate NID2 methylation in a cancer specific manner, in human gastrointestinal cancer; promoter hypermethylation of NID2 was shown in 14 out of 48 colon carcinoma samples analyzed compared to 0/24 normal colon, 19/20 of the gastric carcinomas, and 0/13 normal gastric mucosa. Moreover, Renard et al. performed a pharmacologic unmasking method in four performed a pharmacologic unmasking method in four UCC cell lines, generated a list of candidate methylated genes, and subsequently performed methylation-specific PCR (MSP) in UCC and normal tissue samples. In their study, NID2 showed methylation in 66 out of 91 UCC tissues and 0 out of 39 normal urothelial tissues analyzed. They then analyzed promoter methylation of NID2 and TWIST1 as a panel in urine DNA from UCC patients and controls. This two gene panel detected UCC patients with 90% sensitivity and 93% specificity while the sensitivity and specificity of cytology test in the same cohort were 48% and 96% respectively. When analyzing only LGPUCC, they observed a sensitivity of 80% (training set) and 89% (validation set) compared to 45% and 44% from cytology, with a sensitivity of 94% and 91% compared to cytology's sensitivity of 97% and 95%. In our cohort, cytology data was available for 70 LGPUCC cases, and the cytology sensitivity for LGPUCC was 50%, while the methylation sensitivity was about 79% using our3 gene panel (methylation in either: CCND2, CALCA, and/ or CCNA1), values comparable to the 2 gene panel showed by Renard et al.'s study. It would be interesting to analyze a cohort of urine samples from LGUCC cases for all the 5 genes (CCNA1, CCND2, CALCA, NID2 and TWIST1) and determine the sensitivity and specificity of the test. A prospective study using appropriate controls and number of samples is necessary to determine the clinical utility of these markers. Furthermore, subsequently collected urine samples in follow-up visits need to be tested to determine the marker's usefulness in reducing cystoscopy in follow-up visits.

In our study, we considered any recurrence as presence of recurrence. Due to the limited number of primary LGPUCC samples we were not able to stratify the cases based on length of follow-up time to recurrence. The ultimate goal of this pilot study was to identify markers that could be detected in urine samples from LGPUCC patients obtained during follow-up visits after TURBT in order to reduce the need of performing cystoscopies. An optimal non-invasive molecular test will allow for screening of patients before an invasive procedure, which might also reduce the number of cystoscopies necessary in surveillance of non-muscle-invasive bladder cancer. If the test has high sensitivity and specificity, cystoscopy would only be performed in patients who are positive for the non-invasive test.

We focused on determining the feasibility of the detection of cancer specific methylation of three genes by testing urine from UCC cases and controls. These 3 genes (CCND2, CCNA1 and CALCA) were selected from our panel of 8 genes that were analyzed in non-recurrent and recurrent primary LGPUCC. Our findings support that the presence of cancer can be determined by testing the promoter methylation of these genes with high specificity in urine. To our best knowledge, these 3 genes had not been tested previously in LGPUCC urine samples by our group and others; and can be incorporated in a gene panel for future early detection and monitoring of LGPUCC patients. We analyzed 148 urine samples, and of the 125 with known grade, 101 of those urine samples were collected from LGPUCC patients. 97 of 101 LGPUCC cases were methylation positive for at least one of the 3 markers tested. Interestingly, our methylation assays were able to detect 25 LGPUCC cases where urine cytology was negative. The latter suggests that these markers may have potential for non-invasive monitoring of LGPUCC after TURBT. Due to the limited amount of bisulfite converted DNA, we were not able to assess NID2 methylation in urine DNA of UCC cases and controls. However, this gene has previously shown excellent discrimination between urine of UCC patients and controls, with a sensitivity of 94% and a specificity of 91% [14].

We tested the relevance of promoter methylation compared to expression of two members (CCNA1 and CCND2) of the cyclin family in this study and in general methylation was correlated with expression in UCC cell lines. CCNA1 is known to be a downstream target of TP53 [32], and CCNA1 methylation was shown to be inversely related to p53 mutational status in primary Head and Neck Squamous cell carcinomas (HNSCC). Forced expression of CCNA1 resulted in robust induction of wild-type p53 in HNSCC cell lines [16]. CCNA1 is frequently inactivated in UCC [11], which indicates its anti-proliferative activity; however, in a recent study, it has been implicated that CCNA1 contributes to prostate cancer invasion and metastasis [33]. It may be speculated that CCNA1 may play different roles in different tumor types and in different biological contexts. Our data in non-recurrent and recurrent primary LGPUCC demonstrated that CCNA1 is significantly more methylated (e.g. silenced) in recurrent LGPUCC than in non-recurrent LGPUCC.

All of the remaining studied genes have been previously described as hypermethylated in UCC: CALCA (calcitonin-related polypeptide alpha is involved in calcium regulation and acts to regulate phosphorus metabolism) was not only shown to have a UCC specific methylation pattern, but was also correlated to later stage tumors (>pT2) [11]. ARF or p14, an important player in cell cycle regulation, has been previously studied in UCC, and the range of methylation frequency observed was between 0 and 56% [44, 45]. Dominguez et al. [45] showed that the presence of p14 methylation in the plasma was significantly associated to recurrence in UCC. In our cohort, we could not confirm this data in tumor samples, which may be due to the limited sample size. RAR-β2, involved in cell differentiation, has been analyzed in UCC to give diverse results, from 2 to almost 90% methylation [46, 47]. Promoter methylation of TIMP3 (tissue inhibitor of metalloproteinases-3) in urine DNA was shown to be an independent prognostic factor for UCC [13]; however, here, we did not observe a correlation with recurrence in primary LGPUCC samples. An extended study using a larger primary LGPUCC cohort will elucidate the role of TIMP3 in recurrence of LGPUCC.

In summary, this work not only sheds light onto new potential methylation based markers associated with recurrent LGPUCC, but also shows the potential of detection of 3 novel genes in urine sediments and demonstrates initial evidence of tumor suppressive activities of CCNA1 in the context of the biology of UCC cell lines.

References noted in the Examples section and throughout can be found in Maldonado et al., www.impactjournals.com/oncotarget (Vol. 5, No. 14, (2014), pp.5218-5233), which is herein incorporated by reference in its entirety.

Although the invention has been described with reference to the above example, it will be understood that modifications and variations are encompassed within the spirit and scope of the invention. Accordingly, the invention is limited only by the following claims.

1. A method for detecting unmethylated cytosine in the promoter of a target gene comprising:
a) contacting a nucleic acid sample from a subject having or at risk of having a urothelial cell proliferation disorder with a bisulfite preparation, thereby modifying unmethylated cytosine to uracil,
b) detecting within the promoter region of one or more of the target genes selected from ARF, TIMP3, RAR-β2, NID2, CCNA1, AIM1, CALCA,CCND2 or any combination thereof, a change in the ratio of cytosine to uracil,
wherein, an increase in uracil content of the nucleic acid is indicative of unmethylated cytosine in the promoter of the target gene.

2. A method for detecting a methylation state of a target gene comprising:
a) contacting a nucleic acid sample from a subject having or at risk of having a urothelial cell proliferation disorder with a methylation sensitive nucleic acid cleavage composition, thereby generating nucleic acid fragments as cleavage product,
b) determining the nucleic acid fragments based on cleavage within the promoter region of a target gene selected from ARF, TIMP3, RAR-β2, NID2, CCNA1, AIM1, CALCA, CCND2, or any combination thereof, wherein a change in the ratio of fragmented to unfragmented products due to cleavage within the promoter region of the gene is indicative of the methylation state of the promoter of the target gene.

3. The method claim 1, further comprising determining the location of the site within the promoter of the target gene which is methylated in the subject sample.

4. The method of claim 1 or 2, wherein the sample is from a human.

5. The method of claim 1 or 2, wherein the sample is selected from the group consisting of a biopsy specimen, a tissue specimen, ejaculate, urine and blood.

6. The method of claim 2, wherein the methylation sensitive cleavage compound is a restriction endonuclease, which is selected from the group consisting of MspI, HpaII, BssHII, BstUI and NotI.

7. The method of claim 1, further comprising after contacting the target gene with bisulfite, contacting the target gene with a probe having homology with the target gene and determining a mismatch between the probe sequence and the contacted nucleic acid within the promoter of the target gene, wherein the mismatch indicates the methylation state of the target gene.

8. The method of claim 2, further comprising, after contacting the nucleic acid with the nucleic acid cleavage composition, contacting the nucleic acid with a probe having homology within the target gene and determining mismatch between the probe sequence and the contacted nucleic acid within the promoter of the target gene, wherein the mismatch indicates the methylation state of the promoter.

9. The method of claim 7, wherein the reagent is a nucleic acid probe.

10. The method of claim 9, wherein the probe is detectably labeled.

11. The method of claim 10, wherein the label is selected from the group consisting of a radioisotope, a bioluminescent compound, a chemiluminescent compound, a fluorescent compound, a metal chelate, and an enzyme.

12. The method of claim 7, wherein the probe comprises a fragment of the promoter region of ARF, TIMP3, RAR-β2, NID2, AIM, CCND2, CCNA1 or CALCA genes.

13. The method of claim 1, further comprising a nucleic acid amplification step prior to the detection.

14. The method of claim 13, wherein the detecting comprises amplifying CpG-containing nucleic acids by means of CpG-specific oligonucleotide primers, wherein the oligonucleotide primers distinguish between modified methylated and non-methylated nucleic acid, and detecting the methylated CpG-containing promoter region based on the presence or absence of amplification products produced in the amplifying step.

15. A method for monitoring the effectiveness of a therapeutic regimen based on recurrence in a subject having a urothelial cell proliferative disorder associated with CCND2, CCNA1 or CALCA promoter hypermethylation comprising:
contacting the subject's nucleic acid sample with a reagent which detects CCND2, CCNA1 or CALCA, wherein the reagent detects methylation state of the regulatory region of CCND2, CCNA1 or CALCA, wherein the regulatory region is the promoter,
contacting the subject's nucleic acid sample with reagents which detect the CCND2, CCNA1 or CALCA RNA level in the sample, wherein a reduction of hypermethylation of the promoter of CCND2, CCNA1 or CALCA DNA, as compared to prior to treatment, or increased levels of CCND2, CCNA1 or CALCA RNA, as compared with the level of CCND2, CCNA1 or CALCA RNA prior to treatment, is indicative of effectiveness of a therapeutic regimen for treatment of urothelial cell proliferative disorder in the subject.

16. The method of claim 15, wherein the therapeutic regimen is chemotherapy.

17. The method of claim 16, wherein the chemotherapy is paclitaxel.

18. A method of treating a urothelial cell proliferative disorder associated with hypermethylation within the promoter region of the genes CCND2, CCNA1 or CALCA in a subject comprising:
contacting a CCND2, CCNA1 or CALCA containing nucleic acid sequence in the subject with an agent that reduces methylation of or demethylates the promoter region of CCND2, CCNA1 or CALCA , wherein the promoter region is hypermethylated as compared with a subject not having a urothelial cell proliferative disorder, thereby increasing expression of the CCND2, CCNA1 or CALCA gene and ameliorating the symptoms associated with the disorder.

19. The method of claim 18, wherein the subject is treated with a therapeutic regimen comprising administration of one or more chemotherapeutic agents.

20. The method of claim 18, wherein the chemotherapeutic agent is administered in combination with a demethylating agent.

21. The method of claim 20, wherein the demethylating agent is 5-azacytidine, 5-aza-2-deoxycytidine or zebularine.

22. The method of claim 20, further comprising administering a histone deacetylase inhibitor.

23. The method of claim 22, wherein in the histone deacetylase inhibitor is Trichostatin A.

24. The method of claim 15, wherein the disorder is LGPUCC.

25. The method of claim 1, wherein detection is for the recurrence of UCC.

26. The method of claim 1, wherein detection is performed using a microarray or other solid support.


SEQUENCE LISTING (33 sequences)

SEQ ID NO: 1 - DNA, 25 bp, Homo sapiens
  misc_feature: Forward 5-3 (primer)
tggtgatggaggaggtttagtaagt

SEQ ID NO: 2 - DNA, 30 bp, Homo sapiens
  misc_feature: Probe 5-3 (6-FAM-5′- 3′-6-TAMRA)
accaccacccaacacacaataacaaacaca

SEQ ID NO: 3 - DNA, 27 bp, Homo sapiens
  misc_feature: Reverse 5-3 (primer)
aaccaataaaacctactcctcccttaa

SEQ ID NO: 4 - DNA, 20 bp, Homo sapiens
  misc_feature: Forward 5-3 (primer)
cgcgggtattggatgttagt

SEQ ID NO: 5 - DNA, 24 bp, Homo sapiens
  misc_feature: Probe 5-3 (6-FAM-5′- 3′-6-TAMRA)
gggagcgttgcggattattcgtag

SEQ ID NO: 6 - DNA, 20 bp, Homo sapiens
  misc_feature: Reverse 5-3 (primer)
ccgacccacctatacgaaaa

SEQ ID NO: 7 - DNA, 19 bp, Homo sapiens
  misc_feature: Forward 5-3 (primer)
acgggcgttttcggtagtt

SEQ ID NO: 8 - DNA, 26 bp, Homo sapiens
  misc_feature: Probe 5-3 (6-FAM-5′- 3′-6-TAMRA)
cgactctaaaccctacgcacgcgaaa

SEQ ID NO: 9 - DNA, 20 bp, Homo sapiens
  misc_feature: Reverse 5-3 (primer)
ccgaacctccaaaatctcga

SEQ ID NO: 10 - DNA, 23 bp, Homo sapiens
  misc_feature: Forward 5-3 (primer)
gttttggaagtatgagggtgacg

SEQ ID NO: 11 - DNA, 30 bp, Homo sapiens
  misc_feature: Probe 5-3 (6-FAM-5′- 3′-6-TAMRA)
attccgccaatacacaacaaccaataaacg

SEQ ID NO: 12 - DNA, 19 bp, Homo sapiens
  misc_feature: Reverse 5-3 (primer)
ttcccgccgctataaatcg

SEQ ID NO: 13 - DNA, 18 bp, Homo sapiens
  misc_feature: Forward 5-3 (primer)
tcgcggcgagtttattcg

SEQ ID NO: 14 - DNA, 22 bp, Homo sapiens
  misc_feature: Probe 5-3 (6-FAM-5′- 3′-6-TAMRA)
cgttatggcgatgcggtttcgg

SEQ ID NO: 15 - DNA, 16 bp, Homo sapiens
  misc_feature: Reverse 5-3 (primer)
ccgaccgcgacaaacg

SEQ ID NO: 16 - DNA, 27 bp, Homo sapiens
  misc_feature: Forward 5-3 (primer)
tttgatttaaggatgcgttagagtacg

SEQ ID NO: 17 - DNA, 24 bp, Homo sapiens
  misc_feature: Probe 5-3 (6-FAM-5′- 3′-6-TAMRA)
aatccgccaacacgatcgacccta

SEQ ID NO: 18 - DNA, 25 bp, Homo sapiens
  misc_feature: Reverse 5-3 (primer)
actttctccctaaaaaccgactacg

SEQ ID NO: 19 - DNA, 25 bp, Homo sapiens
  misc_feature: Forward 5-3 (primer)
gcggtttttaaggagttttattttc

SEQ ID NO: 20 - DNA, 24 bp, Homo sapiens
  misc_feature: Probe 5-3 (6-FAM-5′- 3′-6-TAMRA)
acgccgctaccccaaaccttacga

SEQ ID NO: 21 - DNA, 21 bp, Homo sapiens
  misc_feature: Reverse 5-3 (primer)
ctacgaaattccctttacgct

SEQ ID NO: 22 - DNA, 27 bp, Homo sapiens
  misc_feature: Forward 5-3 (primer)
gggattagaattttttatgcgagttgt

SEQ ID NO: 23 - DNA, 22 bp, Homo sapiens
  misc_feature: Probe 5-3 (6-FAM-5′- 3′-6-TAMRA)
tgtcgagaacgcgagcgattcg

SEQ ID NO: 24 - DNA, 20 bp, Homo sapiens
  misc_feature: Reverse 5-3 (primer)
taccccgacgatacccaaac

SEQ ID NO: 25 - DNA, 21 bp, Homo sapiens
  misc_feature: Forward 5-3 (primer)
gcgtcggaggttaaggttgtt

SEQ ID NO: 26 - DNA, 19 bp, Homo sapiens
  misc_feature: Probe 5-3 (6-FAM-5′- 3′-6-TAMRA)
aactcgctcgcccgccgaa

SEQ ID NO: 27 - DNA, 22 bp, Homo sapiens
  misc_feature: Reverse 5-3 (primer)
ctctccaaaattaccgtacgcg

SEQ ID NO: 28 - DNA, 20 bp, Homo sapiens
  misc_feature: Forward 5-3 (primer)
cgtcttcaccaccatggaga

SEQ ID NO: 29 - DNA, 20 bp, Homo sapiens
  misc_feature: Reverse 5-3 (primer)
cggccatcacgccacagttt

SEQ ID NO: 30 - DNA, 19 bp, Homo sapiens
  misc_feature: Forward 5-3 (primer)
ctcctgtctggtgggagga

SEQ ID NO: 31 - DNA, 21 bp, Homo sapiens
  misc_feature: Reverse 5-3 (primer)
tcaggtgttattctggatcag

SEQ ID NO: 32 - DNA, 20 bp, Homo sapiens
  misc_feature: Forward 5-3 (primer)
cgcaagcatgctcagacctt

SEQ ID NO: 33 - DNA, 18 bp, Homo sapiens
  misc_feature: Reverse 5-3 (primer)
ccaccgtcgatgatcgca